(19) United States
(12) Patent Application Publication
(10) Pub. No.: US 2020/0058398 A1
(43) Pub. Date: Feb. 20, 2020

(54) METHOD OF DIAGNOSING CANCER USING MITOCHONDRIAL DNA HETEROGENEITY

(71) Applicant: The United States of America, as represented by the Secretary, Dept. of Health and Human Services, Bethesda, MD (US)

(72) Inventor: David S. Campo, Bethesda, MD (US)

(21) Appl. No.: 16/485,714

(22) PCT Filed: Feb. 13, 2018
(86) PCT No.: PCT/US2018/017962

§ 371 Date: Aug. 13, 2019

Related U.S. Application Data
(60) Provisional application No. 62/459,406, filed on Feb. 15, 2017.

(51) Int. Cl. G16H 50/20; G16B 99/00; C12Q 1/6886

(52) U.S. Cl. CPC G16H 50/20; C12Q 1/6886; G16B 99/00

(57) ABSTRACT

The present invention relates to diagnosing cancer based on measurements of sequence heterogeneity in mitochondrial genomic DNA.

### FIELD OF THE INVENTION

The present invention relates to diagnosing cancer based on measurements of sequence heterogeneity in mitochondrial genomic DNA.

### BACKGROUND OF THE INVENTION

Cancer represents the leading cause of morbidity and mortality worldwide, with approximately 14 million new cases and 8.2 million cancer related deaths in 2012. This number is predicted to rise by approximately 70% over the next two decades [1]. The prognosis of cancer patients depends heavily on both early diagnosis and the frequent monitoring of patient response to treatment [2]. Currently, the standard prognostic procedure for cancer is histological analysis of tissue biopsy. But biopsies have several disadvantages as they are invasive, costly and time-consuming. Only highly trained pathologists can perform histological detection of cancer and grade from sampled tissue. In addition, although generally safe, biopsies may cause complications such as bleeding, infection and accidental injury to adjacent structures [2].

Further improvement of cancer patient care greatly depends on development of accurate, minimally invasive, inexpensive and rapid diagnostic techniques. Recent progress in replacing tumor biopsy with testing blood for disease biomarkers opens a new field of cancer diagnostics (see [2] for a review). The rapid, cheap and non-invasive nature of the “liquid biopsy” is bringing a fundamental change to the cancer care by allowing for a repeat sampling and testing of blood for the early disease detection and effective monitoring of treatment responses [4].

Tumors shed nucleic acids into blood, a phenomenon that was exploited since the early discovery of cancer-related DNA mutations [3]. Screening of the whole human genome, the exome or mitochondrial DNA allows for the detection of mutant DNA species associated with different malignant tumors (see [4] for a review). Detection of tumor DNA circulating in blood provides a direct measure of cancer rather than an indirect assessment of the effects of cancer. However, low concentration of the tumor DNA in blood hampers its use in diagnostics. Recently, ultra-deep sequencing (UDS) has been applied to the efficient detection of the tumor DNA, thus significantly facilitating early cancer detection in asymptomatic individuals. Such mutant DNA species can be detected even at a very low concentration in blood of patients. However, the complex and variable genetic nature of cancer in each patient often hinders the identification of mutations suitable for cancer diagnostics (see [4] for a review). The current paradigm that is guiding the research in the area of “liquid biopsies” can be summarized as follows:

1. Find a multitude of nuclear sequences that are mutated in tumors of a certain cancer type. These targets are located in the whole human genome (3.0E+09 bases) or the exome (8.11E+07 bases). Then search for these sequences in the blood of these same cancer patients. At first, their detection was difficult, but UDS has solved this problem.

2. After these targets are found, they are tested in a new set of individuals, hoping that they have predictive power. However, it is often the case that cancer mutations are idiosyncratic (see [4] for a review).

Accordingly, there is a need for improved methods for diagnosing cancer. Described herein is a method that can accurately distinguish cancer from healthy samples using mitochondrial DNA (mtDNA) genetic heterogeneity profiles, which may be obtained from exome sequencing of blood samples.

### SUMMARY OF THE INVENTION

Provided herein is a method of diagnosing cancer in a patient. The cancer may be a liver cancer, which may be hepatocellular carcinoma. The method may comprise providing a heterogeneity profile of mitochondrial DNA (mtDNA) obtained from a blood sample from a patient. The heterogeneity profile may comprise a level of genetic heterogeneity quantified at one or more nucleotide positions of a mtDNA genome. The method may also comprise classifying the patient heterogeneity profile as cancer-positive or cancer-negative based on the result of a machine learning classifier. The method may also comprise diagnosing the presence or absence of cancer in the patient based on the classification.

The machine learning algorithm may be a random forest, which may have been trained with a data set comprising heterogeneity profiles of mtDNA from positive control subjects diagnosed with the cancer and heterogeneity profiles of mtDNA from negative control subjects diagnosed as not having the cancer. The level of genetic heterogeneity at each nucleotide position of the mtDNA genome for each patient and control subject may be quantified by calculating Shannon entropy. The level of genetic heterogeneity at each nucleotide position may also be quantified by transforming the Shannon entropy level into a Z-score. The heterogeneity profiles may comprise levels of entropy quantified at each nucleotide position of the mtDNA genomes of the patient, the positive control subjects, and the negative control subjects. The mtDNA genomes may be sequenced using next-generation sequencing.

Also provided herein is a system. The system may comprise a memory, and at least one computing device. The computing device may be to obtain heterogeneity profile data including data quantifying a level of genetic heterogeneity at one or more nucleotide positions of mtDNA of a blood sample corresponding to a patient. The computing device may also be to execute a machine learning classifier to analyze the patient heterogeneity profile. The machine learning classifier may have been trained with data including first heterogeneity profile data indicative of presence of a cancer and second heterogeneity profile data indicative of absence of the cancer. The computing device may also, based on the prediction of the machine learning classifier, store in the memory, an indication of presence or absence of the cancer corresponding to the patient.

### BRIEF DESCRIPTION OF THE DRAWINGS

FIG. 1 shows an outline of the pre-processing of sequence files.

FIG. 2 shows an outline of the training of a machine learning classifier.

FIG. 3 shows an example of one of the trees in the Random Forest Classifier. Darker-shaded leaves would classify any sample that ends there as “non-cancer,” while lighter-shaded leaves would classify it as “cancer.”

FIG. 4 shows a flow diagram of the cancer diagnostic method described herein to a new sample.

FIGS. 5A-E show characteristics of LC and NC samples. FIG. 5A. Number of samples. FIG. 5B. Number of reads per sample (Log 10). FIG. 5C. Percentage of the mtDNA genome covered. FIG. 5D. Gender distribution. FIG. 5E. mtDNA lineage distribution.

FIG. 6A shows the importance of each nucleotide position entropy in separating cancer and control samples. Only the sites within the top 1% scores (in grey) were used for machine learning. FIG. 6B shows the distribution of samples.

FIG. 7 shows the accuracy of the Random Forest classifier.

FIG. 8 provides a block diagram of a computing environment that may be used to execute cancer diagnostic methods.

FIG. 9 provides an illustration of a computing device.

FIGS. 10A-C show demographic characteristics of the cancer samples. FIG. 10A. Risk factors. FIG. 10B. Detail of Viral Hepatitis risk factors. FIG. 10C. Neoplasm histological grade. FIG. 10D. Gender.

FIGS. 11A-F show comparisons between tissues of cancer patients. FIG. 11A. Number of reads, all pairwise comparisons have a p value. FIG. 11B. mtDNA average depth. FIG. 11C. mtDNA total entropy. FIG. 11D. Percentage of the mtDNA genome covered. FIG. 11E. Percentage of all reads that map to the mtDNA genome. FIG. 11F. Number of polymorphic sites.

FIGS. 12A-C show tumor-specific sites and variants in different LC patients. FIG. 12A. Percentage of tumor-specific sites that are present in several LC patients. FIG. 12B. Percentage of tumor-specific variants that are present in several LC patients. FIG. 12C. Distribution of tumor-specific sites along the genome.

FIGS. 13A-F show differences between LC and NC samples. FIG. 13A. Average entropy. FIG. 13B. Average entropy over the mtDNA genome. Sliding moving window=201 bp, step=1. FIG. 13C. Percentage of all exome reads tha map to the mtDNA genome. FIG. 13D. Percentage of mtDNA sites with high average entropy. FIG. 13E. Percentage of all reads that map to the mtDNA genome. FIG. 13F. Number of polymorphic sites.

### DETAILED DESCRIPTION

Given that tumors shed nucleic acids into the blood, considerable efforts are being directed to using Ultra-Deep Sequencing (UDS) to detect circulating tumor DNA and provide early cancer detection in asymptomatic individuals. Although UDS technology has enabled the search for these nucleic acids, it still remains an open problem which nucleic acids to look for. The current approach is to identify specific changes in the whole human genome (3.0E+09 bases) or the exome (8.11E+07 bases) that occur in tumors of several patients. After these targets are found, they are tested in a new set of individuals, hoping that they have predictive power. Unfortunately, it is often the case that cancer mutations are idiosyncratic.

The inventor has discovered a method of accurately diagnosing cancer that, surprisingly, is based on mtDNA genetic heterogeneity. It is different from known methods in at least three ways: (1) it limits the search to the small mtDNA genome (16569 bases), which has already been functionally associated with several cancer types; (2) measures the genetic heterogeneity of the mtDNA, rather than specific mutations; and (3) applies machine learning algorithms to create a classifier that better generalizes the prediction to new samples. For one of the methods described herein, the classification accuracy on 464 samples (232 liver cancer, 232 healthy controls) is 99.78%. Further, a better estimate of the generalization power of the method uses 10-fold Cross-Validation, which returned an average of 92.23%. Finally, the classifier yielded an accuracy of 93.08% on a test dataset that was never seen by the classifier.

In conclusion, the methods described herein allow for an accurate, efficient and automatic detection of cancer, and liver cancer in one particular method, based on analysis of mtDNA genetic heterogeneity. This rapid, cheap and non-invasive test could be the base of widely available cancer screening programs.

### 1. Definitions

The terminology used herein is for the purpose of describing particular embodiments only and is not intended to be limiting. As used in the specification and the appended claims, the singular forms “a,” “an” and “the” include plural referents unless the context clearly dictates otherwise.

For recitation of numeric ranges herein, each intervening number there between with the same degree of precision is explicitly contemplated. For example, for the range of 6-9, the numbers 7 and 8 are contemplated in addition to 6 and 9, and for the range 6.0-7.0, the numbers 6.0, 6.1, 6.2, 6.3, 6.4, 6.5, 6.6, 6.7, 6.8, 6.9, and 7.0 are explicitly contemplated.

“mitochondrial DNA” or “mtDNA” means DNA from a mitochondrion in a human cell, which may be from a cancer cell such as a tumor. mtDNA may circulate in the blood of a human, particularly one suffering from a cancer such as a tumor. mtDNA may refer to a portion or all of the genomic DNA from a mitochondrion. The nucleotide position of a mtDNA genome may be based on a reference mtDNA genomic DNA sequence. For example, the reference sequence may be as reported by R. M. Andrews, et al., Reanalysis and revision of the Cambridge reference sequence for human mitochondrial DNA. Nat Genet, 23(2), p. 147 (1999), the contents of which are incorporated herein by reference.

### 2. Method of Diagnosing Cancer

Provided herein is a method of diagnosing a cancer in a patient. The method uses genetic heterogeneity as quantified at one or more nucleotide positions in mtDNA obtained from the patient. The patient's genetic heterogeneity is used to classify the patient as cancer-positive or cancer-negative using a machine learning classifier. The machine learning classifier may be trained with a data set of genetic heterogeneity quantified at the same nucleotide position(s) in mtDNA obtained from positive control subjects diagnosed with the cancer and from negative control subjects diagnosed as not having the cancer. The genetic heterogeneity of mtDNA in the patient and in the negative- and positive-control subjects may be quantified at each nucleotide position of the mtDNA genome. A heterogeneity profile for each individual patient or control subject may comprise genetic heterogeneity quantified at one or more nucleotide positions of the mtDNA genome.

a. Cancer

The cancer may be liver cancer, a cancer associated with Hepatitis C Virus (HCV), or a cancer associated with Human Papilloma Virus (HPV). The liver cancer may be hepatocellular carcinoma. The HCV-associated cancer may be head and neck cancer or B-cell lymphoma. The HPV-associated cancer may be cervical cancer. In particular, the cancer may also be a tumor.

b. Control Subjects

The positive- and negative control subjects may be selected based on one or more specific traits, such as age, gender, ethnicity, or geographic location. The control subjects may also be selected based on their mtDNA sequences having been obtained using the same sequencing method, or exome library preparation method. The control subjects may also be selected based on a risk factor, which may be HCV- or HPV-status. The positive control subjects may selected based on cancer type or grade, which may be a histologic grade.

c. mtDNA

The mtDNA may be obtained from a sample obtained from the patient. The sample may be a blood sample, which may be plasma or serum. The blood sample may comprise circulating mtDNA, which may be from the cancer in the patient.

d. mtDNA Sequencing and Sequence Processing

Genetic heterogeneity may be quantified from mtDNA sequence data that was obtained using a Next-Generation sequencing method, which may be an Ultra-Deep Sequencing method. The sequencing method may be AGILENT SURE SELECT, ILLUMINA TRUESEQ, or NIMBLEGEN SEQCAP EZ. The sequencing method may comprise sequencing the exome for a patient or control subject. Exome sequence data may comprise multiple mtDNA genome sequences of a patient or control subject.

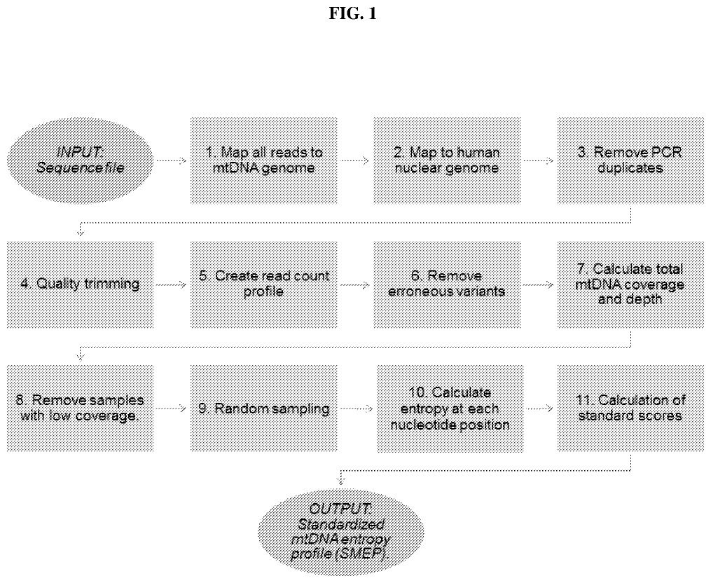

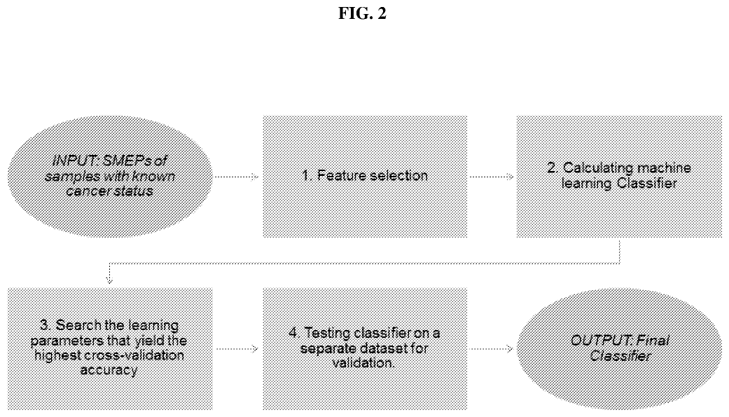

The mtDNA genome sequence data for a patient or control subject may be obtained using an exome sequence processing method known in the art. The processing method may comprise the steps described in FIG. 1. Each exome sequence read from an individual may be mapped to a reference mtDNA genome, which may be done using an approach described by MTOOLBOX (C. Calabrese, et al., MToolBox: a highly automated pipeline for heteroplasmy annotation and prioritization analysis of human mitochondrial variants in high-throughput sequencing. Bioinformatics, 30(21), pp. 3115-7 (2014) and E. Picardi and G. Pesole, Mitochondrial genomes gleaned from human whole-exome sequencing. Nat Methods, 9(6), pp. 523-4 (2012), the contents of both of which are incorporated herein by reference). mtDNA genomic sequence from each exome sequence read may be retained, while all other sequences are discarded. The reference mtDNA genome sequence may be as described by R. M. Andrews, et al., Reanalysis and revision of the Cambridge reference sequence for human mitochondrial DNA. Nat Genet, 23(2), p. 147 (1999), the contents of which are incorporated herein by reference.

The retained mtDNA genome sequence data may then be mapped to a reference nuclear human genome sequence, and nuclear mitochondrial DNA segments may be removed from the mtDNA genome sequence data. PCR duplicates may be removed using a method known in the art, which may be Picard MarkDuplicates (available at broadinstitute.github.io/picard/). Quality trimming may be performed on the mtDNA genome sequence data, which may be by FaQCS (C. Lo, et al., Rapid evaluation and Quality Control of Next Generation Sequencing Data with FaQCs, BMC Bioinformatics, November 19; 15 (2014), the contents of which are incorporated herein by reference). A read count profile may be generated after quality trimming, which may be by using BAM-readCount (available from github.com/genome/bam-readcount). Low frequency variants in mtDNA genome sequences may be distinguished from sequence errors, such as by removing a variant if the probability that the sequence variance was an error is higher than 0.00001. The removal of sequence variants due to sequencing errors may be performed as described by M. J. Morelli, et al., Evolution of foot-and-mouth disease virus intra-sample sequence diversity during serial transmission in bovine hosts. Vet Res, 44:12 (2013), the contents of which are incorporated herein by reference.

The total mtDNA genome coverage and depth at each nucleotide position for a patient or control subject may be calculated. A control subject's sample may be excluded from a machine learning classifier training data set if the sample has a total coverage lower than 99, 98, 97, 96, 95, 94, 93, 92, 91, or 90%.

e. Quantifying Genetic Heterogeneity

The level of genetic heterogeneity may be quantified at one or more nucleotide positions within a mtDNA genome, and may be quantified at each nucleotide position of the mtDNA genome. Genetic heterogeneity may be quantified from mtDNA genome sequences of an individual that have been processed as described herein. It may also be quantified from multiple random samples of multiple mtDNA genome sequence reads from a patient or control subject. The quantification may be performed on at least 20, 30, 40, 50, 60, 70, 80, 90, or 100 random samples of mtDNA sequences from an individual, with replacement of at least 50, 100, 150, 200, or 250 reads per position, which may be followed by an average over all random samples. The number of reads may be based on the average depth found in a particular mtDNA genome sequence dataset.

Genetic heterogeneity at a nucleotide position within the mtDNA may be quantified by measuring entropy. In particular, the Shannon entropy may be measured, which may be according to the following formula:

$${H}_{j} = - {\sum }_{i = 1}^{n} {x}_{i} {log}_{b} {x}_{i}$$

where xᵢ is the fraction of reads covering that position that show variant i and b is the base of the logarithm, where in one example b=2.

Quantifying the entropy level may also comprise normalization using a method known in the art. For example, normalization may comprise transforming an entropy level into a Z-score, which may comprise the signed number of standard deviations by which each observation is above or below the mean of the sample. Measurements of mtDNA genetic heterogeneity (for example, entropy levels, or normalized entropy levels which may be transformed into Z-scores) for an individual at one or more nucleotide positions of a mtDNA genome sequence may be included in a heterogeneity profile.

f. Machine Learning Algorithm

A machine learning classifier is used to classify a patient's sample as positive or negative for the cancer. The machine learning classifier may be trained using, or may comprise, heterogeneity profiles from positive- and negative-control subjects. The machine learning algorithm used in the classifier may be Support Vector Machines, K-Nearest Neighbor, Nearest Centroid, Logistic Regression, Naïve Bayes, Decision Trees, or Random Forest. In particular, the machine learning classifier may be Random Forest, which may be trained using heterogeneity profiles of positive- and negative-control subjects.

The machine learning classifier may be trained by a method comprising steps described in FIG. 2. Training the machine learning classifier may comprise selecting a subset of particular nucleotide positions of the mtDNA genome for use in the classifier, which may comprise using a method known in the art such as ReliefF (M. Robnik-Sikonja and I. Kononenko, Theoretical and Empirical Analysis of ReliefF and RReliefF. Machine Learning Journal, 53: pp. 23-69 (2003), the contents of which are incorporated herein by reference). Training may also comprise performing supervised learning by means of the machine learning algorithm. For example, the learning may be performed using Random Forest, as implemented in a method known in the art, which may be Scikit (F. Pedregosa, et al., Scikit-learn: Machine learning in Python. Journal of Machine Learning Research, 12(October): pp. 2825-2830 (2011), the contents of which are incorporated herein by reference).

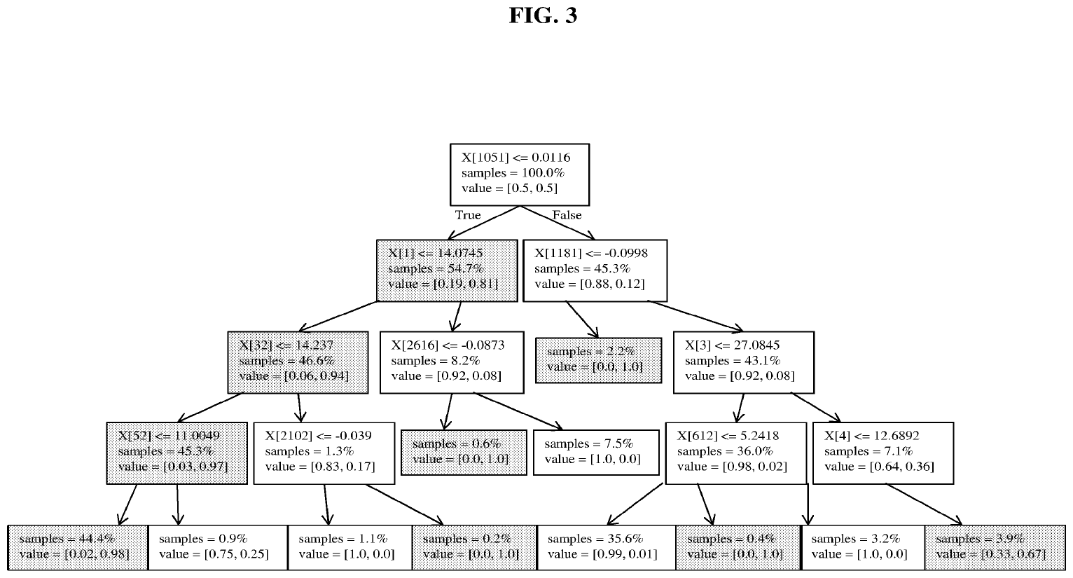

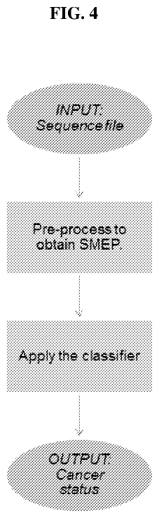

Training the machine learning classifier may also comprise performing a grid search of the best combination of parameters. The grid search may comprise performing 10-fold cross validation. The parameters used in the machine learning classifier may comprise the number of trees, maximum tree depth, minimum number of instances to perform a split, splitting criterion, minimum number of instances in a leaf, and class weight. In one example, the number of trees may be 101, the maximum tree depth may be 4, the minimum number of instances to perform a split may be 19, the splitting criterion may be entropy, the minimum number of instances in a leaf may be 1, and the class weight may be balanced. The machine learning classifier may be selected to have the highest cross-validation accuracy among multiple machine learning classifiers tested. For example, a liver cancer classifier may comprise a tree as described in FIG. 3, where “X [#]” indicates the nucleotide position within the mtDNA genome (for example, position 1051 in the highest node in the tree), the value next to the nucleotide position (for example, “<=0.0116” in the highest node in the tree) indicates the threshold entropy level, “samples” indicates the percentage of samples falling into the node, and “value [#,#]” indicates the proportion of control subjects with the cancer in the first bracketed value, and the proportion of non-cancer subjects in the second bracketed value (for example, 0.5 cancer and 0.5 non-cancer in the highest node in the tree).

g. Classifying a Patient Sample

The machine learning classifier may be applied to the mtDNA genetic heterogeneity of the patient, such as in the patient's heterogeneity profile. Classifying the patient's heterogeneity profile as cancer-positive or cancer-negative may comprise the steps described in FIG. 4. As described herein, the patient's heterogeneity profile may comprise the level of genetic heterogeneity quantified at one or more nucleotide positions of the mtDNA genome. The patient's exome sequences may have been processed as described herein before determining the patient's heterogeneity profile. The machine learning classifier may classify the patient's heterogeneity profile as cancer-positive or cancer-negative. The classification may comprise a prediction confidence threshold, wherein if the heterogeneity profile is above the threshold then the profile is classified as cancer-positive, and if the heterogeneity profile is below the threshold then the profile is classified as cancer-negative. For example, if the machine learning classifier is Random Forest, and the majority of trees classify the patent's heterogeneity profile as cancer-positive, then the presence of the cancer may be diagnosed in the patient.

### 3. Systems

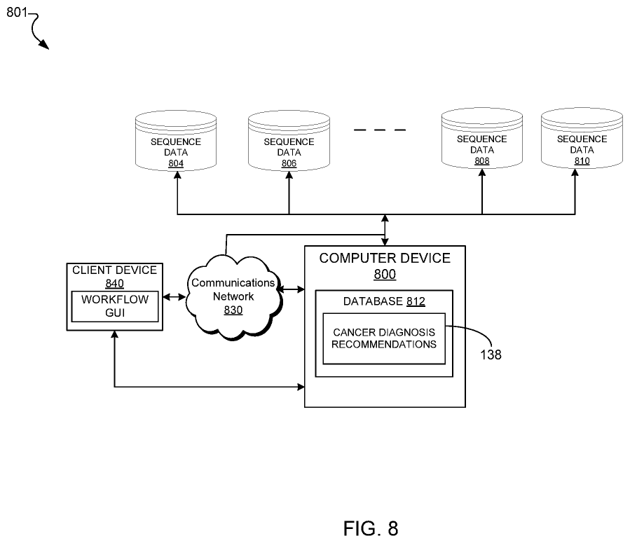

FIG. 8 provides an illustration of a computing system 801 for automatically generating cancer diagnosis recommendations based on measurements of sequence heterogeneity in mtDNA. More specifically, FIG. 8 illustrates a computer device 800 (e.g., a server device) that implements and/or otherwise executes various processes for analyzing sequence data to generate recommendations defining or otherwise indicating a diagnosis of a specific type of cancer. Generally speaking, sequence data represents a type of biological data that is composed of a large collection of computerized (“digital”) nucleic acid sequences, protein sequences, and/or other polymer sequences that may be maintained in a data base, data store, storage appliance, memory, or some other type of storage capacity. In one specific example, the sequence data may include or otherwise be equivalent to mtDNA sequence data, as described above in section 2(d). As illustrated in FIG. 8, the sequence data may be stored or otherwise maintained in various databases 804-810, each of which may be a structured and/or unstructured data source.

The computing device 800 may receive or otherwise extract sequence data from the databases 804-810 and reconcile the received sequencing data into a single dataset. The computing device 800 may then generate one or more outputs 812, in the form of a recommendation or diagnosis of cancer 138, which may be provided to users, such as a physician, for assistance in the areas of screening, diagnosing and/or staging of cancer.

The computing device 800 may employ various machine learning methodologies to enable the computing device 800 to automatically and continuously learn to analyze the sequence data and automatically and continuously generate more accurate cancer diagnosis recommendations. Generally speaking, machine learning represents a form of computing in which artificial intelligence is employed to allow computers to evolve behaviors based on empirical data. Machine learning may take advantage of training examples to capture characteristics of interest of its unknown underlying probability distribution. Training data may be seen as examples that illustrate relations between observed variables. In one specific example, the machine learning techniques automatically executed by the computing device 800 may be the machine learning algorithms described above in section 2(f), although it is contemplated that other machine learning algorithms may be used. The computing device 800 may include or otherwise execute its machine learning algorithms based on the training data described in Example 1, “Training of the Machine Learning Classifier.”

The computer device 800 functionally connects (e.g., using communications network 830) to one or more client devices 840 included within the computing network 801. The one or more client devices 840 may service the need of users interested in generating cancer diagnosis recommendations. To do so, a user may interact with the one or more of the client device 840 and provide input, which may be processed by the computing device 800. The one or more client devices 840 may be any of, or any combination of, a personal computer; handheld computer; mobile phone; digital assistant; smart phone; server; application; wearable, IOT device and the like. In one embodiment, each of the one or more client devices 840 may include a processor-based platform that operates on any suitable operating system, such as Microsoft® Windows®, Apple OSX®, Linux®, and/or the like that is capable of executing software. The computing network 801 may be an IP-based telecommunications network, the Internet, an intranet, a local area network, a wireless local network, a content distribution network, or any other type of communications network, as well as combinations of networks.

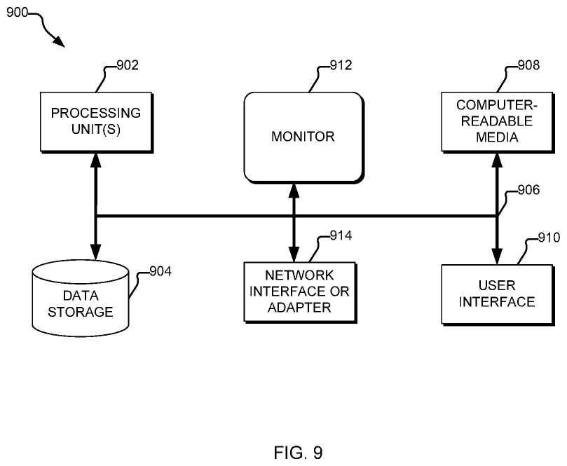

FIG. 9 illustrates an example of a suitable computing and networking environment 900 that may be used to implement various aspects of the present disclosure described in FIGS. 8 and 9 (e.g. the computing device 800 and corresponding components). As illustrated, the computing and networking environment 900 includes a general purpose computing device 900, although it is contemplated that the networking environment 900 may include other computing systems, such as personal computers, server computers, hand-held or laptop devices, tablet devices, multiprocessor systems, microprocessor-based systems, set top boxes, programmable consumer electronic devices, network PCs, minicomputers, mainframe computers, digital signal processors, state machines, logic circuitries, distributed computing environments that include any of the above computing systems or devices, and the like.

Components of the computer 900 may include various hardware components, such as a processing unit 902, a data storage 904 (e.g., a system memory), and a system bus 906 that couples various system components of the computer 900 to the processing unit 902. The system bus 906 may be any of several types of bus structures including a memory bus or memory controller, a peripheral bus, and a local bus using any of a variety of bus architectures. For example, such architectures may include Industry Standard Architecture (ISA) bus, Micro Channel Architecture (MCA) bus, Enhanced ISA (EISA) bus, Video Electronics Standards Association (VESA) local bus, and Peripheral Component Interconnect (PCI) bus also known as Mezzanine bus.

The computer 900 may further include a variety of computer-readable media 908 that includes removable/non-removable media and volatile/nonvolatile media, but excludes transitory propagated signals. Computer-readable media 908 may also include computer storage media and communication media. Computer storage media includes removable/non-removable media and volatile/nonvolatile media implemented in any method or technology for storage of information, such as computer-readable instructions, data structures, program modules or other data, such as RAM, ROM, EEPROM, flash memory or other memory technology, CD-ROM, digital versatile disks (DVD) or other optical disk storage, magnetic cassettes, magnetic tape, magnetic disk storage or other magnetic storage devices, or any other medium that may be used to store the desired information/data and which may be accessed by the computer 900. Communication media includes computer-readable instructions, data structures, program modules or other data in a modulated data signal such as a carrier wave or other transport mechanism and includes any information delivery media. The term “modulated data signal” means a signal that has one or more of its characteristics set or changed in such a manner as to encode information in the signal. For example, communication media may include wired media such as a wired network or direct-wired connection and wireless media such as acoustic, RF, infrared, and/or other wireless media, or some combination thereof. Computer-readable media may be embodied as a computer program product, such as software stored on computer storage media.

The data storage or system memory 904 includes computer storage media in the form of volatile/nonvolatile memory such as read only memory (ROM) and random access memory (RAM). A basic input/output system (BIOS), containing the basic routines that help to transfer information between elements within the computer 900 (e.g., during start-up) is typically stored in ROM. RAM typically contains data and/or program modules that are immediately accessible to and/or presently being operated on by processing unit 902. For example, in one embodiment, data storage 904 holds an operating system, application programs, and other program modules and program data.

Data storage 904 may also include other removable/non-removable, volatile/nonvolatile computer storage media. For example, data storage 904 may be: a hard disk drive that reads from or writes to non-removable, nonvolatile magnetic media; a magnetic disk drive that reads from or writes to a removable, nonvolatile magnetic disk; and/or an optical disk drive that reads from or writes to a removable, nonvolatile optical disk such as a CD-ROM or other optical media. Other removable/non-removable, volatile/nonvolatile computer storage media may include magnetic tape cassettes, flash memory cards, digital versatile disks, digital video tape, solid state RAM, solid state ROM, and the like. The drives and their associated computer storage media, described above and illustrated in FIG. 9, provide storage of computer-readable instructions, data structures, program modules and other data for the computer 900.

A user may enter commands and information through a user interface 910 or other input devices such as a tablet, electronic digitizer, a microphone, keyboard, and/or pointing device, commonly referred to as mouse, trackball or touch pad. Other input devices may include a joystick, game pad, satellite dish, scanner, or the like. Additionally, voice inputs, gesture inputs (e.g., via hands or fingers), or other natural user interfaces may also be used with the appropriate input devices, such as a microphone, camera, tablet, touch pad, glove, or other sensor. These and other input devices are often connected to the processing unit 902 through a user interface 910 that is coupled to the system bus 906, but may be connected by other interface and bus structures, such as a parallel port, game port or a universal serial bus (USB). A monitor 912 or other type of display device is also connected to the system bus 906 via an interface, such as a video interface. The monitor 912 may also be integrated with a touch-screen panel or the like.

The computer 900 may operate in a networked or cloud-computing environment using logical connections of a network interface or adapter 914 to one or more remote devices, such as a remote computer. The remote computer may be a personal computer, a server, a router, a network PC, a peer device or other common network node, and typically includes many or all of the elements described above relative to the computer 900. The logical connections depicted in FIG. 9 include one or more local area networks (LAN) and one or more wide area networks (WAN), but may also include other networks. Such networking environments are commonplace in offices, enterprise-wide computer networks, intranets and the Internet.

When used in a networked or cloud-computing environment, the computer 900 may be connected to a public and/or private network through the network interface or adapter 914. In such embodiments, a modem or other means for establishing communications over the network is connected to the system bus 906 via the network interface or adapter 914 or other appropriate mechanism. A wireless networking component including an interface and antenna may be coupled through a suitable device such as an access point or peer computer to a network. In a networked environment, program modules depicted relative to the computer 900, or portions thereof, may be stored in the remote memory storage device.

The present invention has multiple aspects, illustrated by the following non-limiting examples.

### Example 1

### Diagnosing Liver Cancer by Measuring mtDNA Heterogeneity Profiles

This example demonstrates that heterogeneity profiles of the intra-host mtDNA population are strongly associated with liver cancer (LC), and thus can be used to diagnose liver cancer. The small size of mtDNA is especially suitable for the accurate assessment of such profiles, application of which to the LC detection overcomes the often-idiosyncratic association of specific mutations to cancer. Genetic diversity of intra-host mtDNA in blood may be used as a generalizable marker for accurate, rapid, inexpensive and minimally invasive diagnostic detection of cancer.

Pre-Processing

FIG. 1 shows the bioinformatics pipeline implemented for pre-processing sequence files. The input was an exome sequence file in fastq format, and the output was a standardized mtDNA entropy profile (SMEP).

(1) The first step was to map all the reads to the mtDNA reference genome [5], using the recommendations and parameters implemented in MTOOLBOX [6, 7]. Reads that mapped to the mtDNA genome were retained, whereas all others were discarded.

(2) Those reads that mapped to the mtDNA were then mapped to the nuclear human genome in order to remove NUMTs (nuclear mitochondrial DNA segments) following the recommendations and parameters of [6, 7].

(3) PCR duplicates were removed with Picard MarkDuplicates (broadinstitute.github.io/picard/)

(4) Quality trimming was performed with FAQCS [8].

(5) A read count profile was created with BAM-readCount (github.com/genome/bam-readcount).

(6) Low frequency variants were distinguished from Illumina sequence errors following [9]. A variant was removed if the probability that it was an error was higher than 0.00001.

(7) The total mtDNA coverage and the depth at each position were calculated.

(8) Those samples with a total coverage lower than 95% of the mtDNA genome are removed from the analysis.

(9) In order to reduce differences in genetic heterogeneity among files that were solely due to sampling depth, 100 random samples of 50 reads were taken at each mtDNA position. The target number of reads was chosen as this was the average depth found in the Liver cancer dataset (n=49.6).

(10) The genetic heterogeneity of each nucleotide position for each of the 100 random samples was calculated using Shannon entropy[10], followed by an average of the entropy values over all random samples. The Shannon entropy H of a nucleotide position j with n different variants is given by:

$${H}_{j} = - {\sum }_{i = 1}^{n} {x}_{i} {log}_{b} {x}_{i}$$

Where xᵢ is the fraction of reads covering that position that show variant i and b is the base of the logarithm (in this case, b=2).

(11) Finally, to make the profiles were more comparable and increase the generalization power of the test, each heterogeneity profile was transformed into a set of Z-scores (also known as standard scores), the signed number of standard deviations by which each observation is above or below the mean of the sample. This standardization greatly improved the accuracy of the classifier.

Training of the Machine Learning Classifier

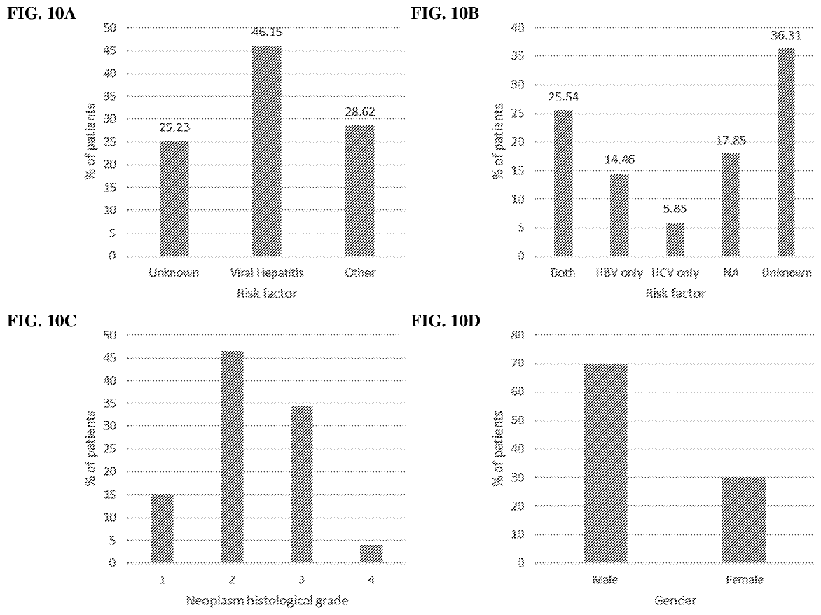

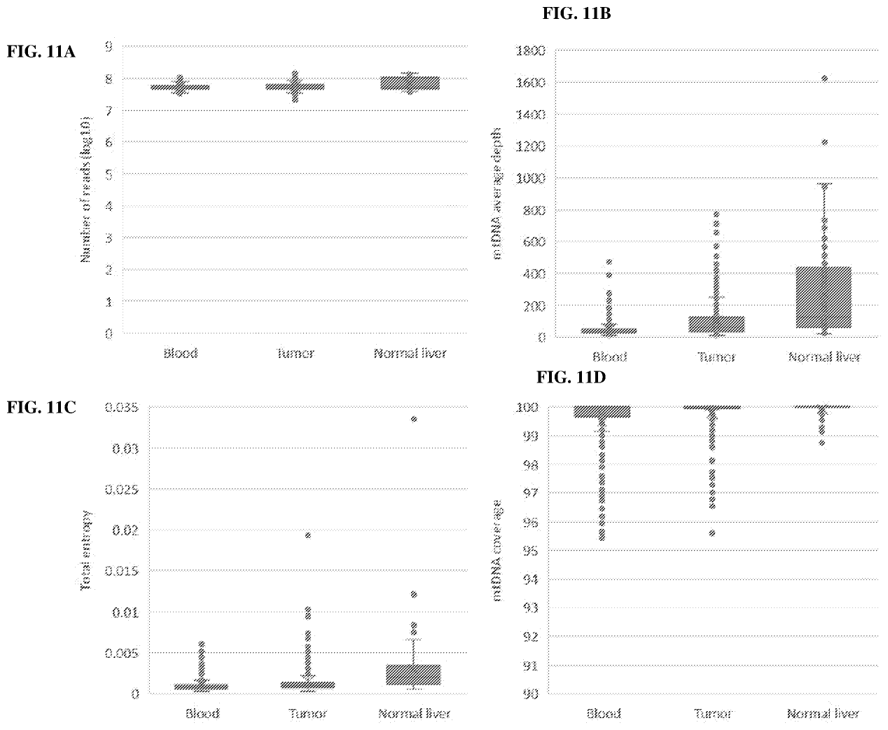

A dataset of samples was defined with known liver cancer status. The Cancer Genome Atlas (TCGA) Research Network [11] had Illumina exome data from 11079 patients and 34 different cancer types, including 376 patients with Liver Hepatocellular Carcinoma. FIG. 10 shows demographic characteristics of the liver cancer samples. Regarding the neoplasm histologic grade, 41 samples were stage 1, 134 samples were stage 2, 104 samples were stage 3, and 13 samples were stage 4. For healthy non-cancer controls (NC), samples from the 1000 Genomes project were obtained [12]. This project held UDS data for 2504 individuals from 26 human populations. From these, 293 samples were selected that satisfied the following criteria: (i) Were unrelated to each other; (ii) Same geographic location of population source as the Cancer samples; (iii) Same technology (Illumina) and Same exome library preparation as the Cancer samples (Nimblegen); (iv) mtDNA genome coverage higher than 95% and (v) the total set matched the gender and mtDNA lineage distribution of the cancer samples.

After defining the training dataset, each sample was pre-processed as described above to obtain its SMEP. FIG. 2 shows the outline of the training process, starting with the SMEP and ending with a final classifier.

(1) First, a subset comprised of the best nucleotide positions were chosen by feature selection with ReliefF [13]. In this case, only the top 1% nucleotide positions were used in separating cancers vs controls.

(2) Supervised machine learning was performed by means of the Random Forest technique[14], as implemented in SCI-KIT [15]. Although other methods were also studied (Nearest Neighbors, Nearest centroid, Support Vector Machine, Logistic regression, Gaussian Naïve Bayes, Decision trees and a Perceptron), Random Forest consistently provided the best results.

(3) A grid search of the best combination of parameters was performed. The performance of each combination of parameters was measured by means of a 10-fold Cross validation. The final parameters of the classifier were the following: Number of trees: 100; Maximum tree depth: 4; Minimum number of instances to perform a split: 19. Splitting criterion: entropy; minimum number of instances in a leaf: 1; class weight: balanced.

(4) Finally, the classifier with the highest Cross-validation accuracy was used to test a dataset that was never seen during the parameter optimization. One of the trees of the Random Forest Classifier is shown in FIG. 3.

Sequencing of Hyper-Variable Segment 1 (HVS1) from mtDNA

Samples:

Blood samples were tested under the CDC ethical guidance. Samples were collected from 136 unrelated patients infected with HCV and with unknown cancer status.

Nucleic Acid Extraction:

Total nucleic acid was extracted from serum samples using the automated Roche MagNA Pure LC robot and the MagNA Pure LC Total Nucleic Acid Isolation kit (Roche Diagnostics, Indianapolis, Ind.), and eluted with 50 μl of elution buffer according to the manufacturer's instructions.

mtDNA Amplification:

One set of primers was used to amplify HVS1: forward primer: HVS1-L15997 (CAC CAT TAG CAC CCA AAG CT) (SEQ ID NO: 1) and reverse primer: HVS1-H16391 (GAG GAT GGT GGT CAA GGG AC) (SEQ ID NO: 2). These primers contain the MiSeq chemistry-specific sequences, p5, p′7, i5, and i7, in addition to molecular identification sequences (MID). PCR was prepared using Perfecta SYBR Green (Quanta Biosciences, Gaithersburg, Md.). The following PCR cycling conditions were used: 95° C. for 5 min, 40 cycles of 95° C. for 30 sec, 55° C. for 30 sec and 72° C. for 1 min, followed by a hold at 72° C. for 7 min before an infinite hold at 4° C. Following the PCR, the target amplicon was quantified using Agilent 2200 TapeStation System (Agilent Technologies Inc., Santa Clara, Calif.) prior to normalizing and pooling the samples into a library for sequencing. PCR and TapeStation plate preparations, sequencing library normalization, and subsequent sample pooling were all performed using the Biomek 3000 liquid handler (Beckman Coulter, Indianapolis, Ind.) in order to automate and standardize the protocol, and to reduce chances of human error.

UDS:

PCR products were pooled and sequenced using the Illumina MiSeq instrument and MiSeq Reagent Kit v3 (600-cycle) (Illumina Inc., San Diego, Calif.).

Application of the Classifier

FIG. 4 briefly shows the outline of an application of the classifier to a new sample of unknown cancer status, the Exome sequencing file is pre-processed to obtain the SMEP, which in turn is passed to the classifier to obtain the prediction confidence that the sample comes from an individual with liver cancer. The prediction confidence is the fraction out 100 trees where the sample was classified as cancer. The classifier is implemented in Python and has been optimized to run in a Linux cluster, taking an average of 30 minutes per set of 16 new samples.

Results

mtDNA from Liver and Blood of LC Patients

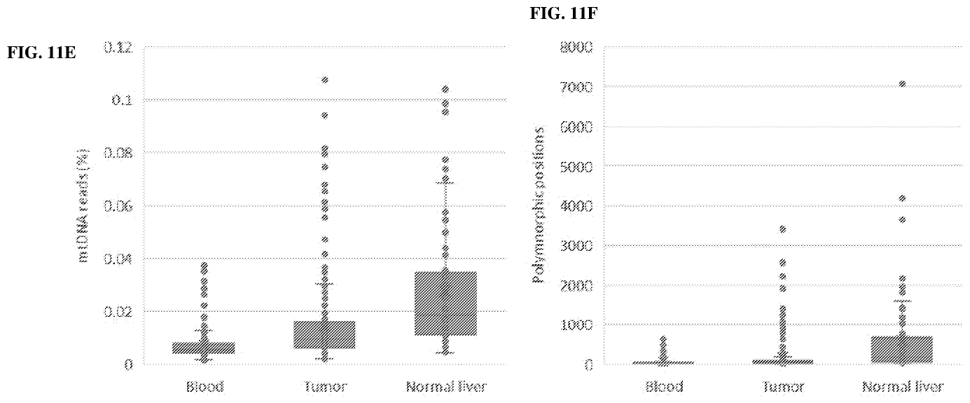

Three samples of tissue were available from LC patients: tumor (n=358), normal liver (n=85) and blood (n=293). FIG. 11 shows comparison among the samples' average number of reads, average depth of mtDNA sequencing, total mtDNA entropy, percentage of the mtDNA genome covered, percentage of all reads that map to mtDNA, and number of polymorphic positions. Pairwise comparison among 3 tissues in each LC patient showed that, with exception of the number of reads, the above parameters are significantly higher in normal liver (paired t-test; p<0.05), while the lowest values were detected in blood (Table 1), indicating a lower representation of mtDNA in blood as compared to liver and reduction in mtDNA in tumor as compared to normal liver.

TABLE 1
Comparison between tissues of LC patients
Ratio of the averages and p value of the paired samples t-test.

| | Blood vs | | | | Normal | |
|---|---|---|---|---|---|---|
| | Normal | | Blood vs | | liver vs | |
| | liver | p value | Tumor | p value | Tumor | p value |
| Number of | 25 | N/A | 277 | N/A | 81 | N/A |
| patients | | | | | | |
| Number of reads | 1.02 | 8.78E−01 | 0.99 | 7.82E−01 | 1.02 | 7.82E−01 |
| (log10) | | | | | | |
| mtDNA average | 0.14 | 9.96E−05 | 0.49 | 8.30E−12 | 1.86 | 6.68E−04 |
| depth | | | | | | |
| mtDNA total | 0.49 | 1.40E−03 | 0.75 | 2.22E−03 | 1.26 | 2.44E−01 |
| entropy | | | | | | |
| Percentage of the | 0.98 | 1.06E−06 | 1.00 | 1.95E−06 | 1.00 | 4.87E−04 |
| mtDNA genome | | | | | | |
| covered | | | | | | |
| Percentage of all | 0.19 | 1.45E−06 | 0.50 | 2.02E−12 | 1.72 | 3.33E−04 |
| reads that map to | | | | | | |
| the mtDNA | | | | | | |
| genome | | | | | | |
| Number of | 0.18 | 3.17E−04 | 0.50 | 8.29E−04 | 2.09 | 2.52E−03 |
| polymorphic | | | | | | |
| sites. | | | | | | |
| Number of | 468 | N/A | 319 | N/A | 492 | N/A |
| different sites | | | | | | |
| (p < 0.05) | | | | | | |

Consensus sequences of mtDNA were generated for each tissue in each samples. On average, the consensus sequences of mtDNA found in tumors and blood of same patient differ at 0.92 sites, being identical in 42.23% of the patients. Consensus sequences from tumor and normal tissue differ at 1.17 sites, being identical in only 37.04% of the individuals. Consensus sequences from blood and normal liver tissue of same patient are much more similar, with an average difference at 0.16 sites and consensus sequences being identical in 84% of patients.

Pairwise comparison of UDS data from the three tissue samples identified 492 sites, entropy of which was significantly different between tumor and normal liver (paired t-test; p<0.05). However, only 38 of the sites differ between tumor and blood (paired t-test; p<0.05), while blood and tumor mtDNA differ at 319 sites (p<0.05). Despite significant similarity of consensus sequences, entropy of 468 sites differ in mtDNA from blood and normal liver (p<0.05), indicating differences in intra-host mtDNA heterogeneity between these two tissues.

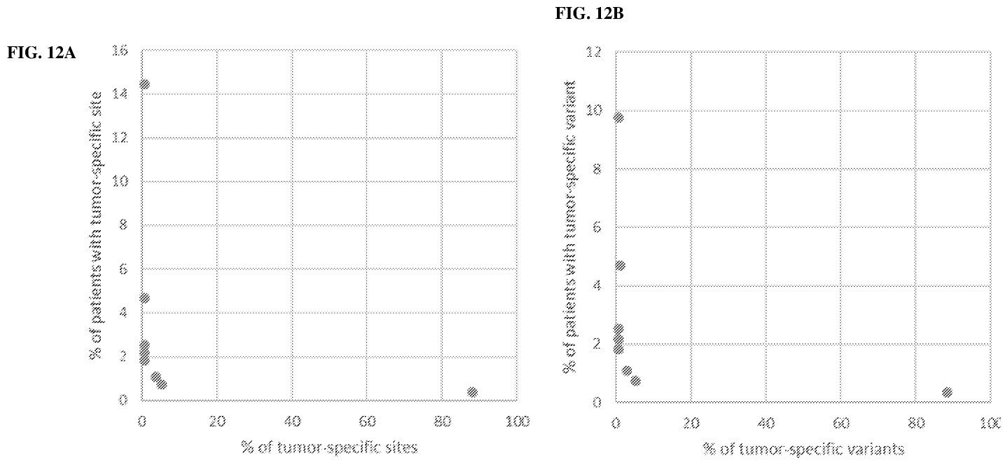

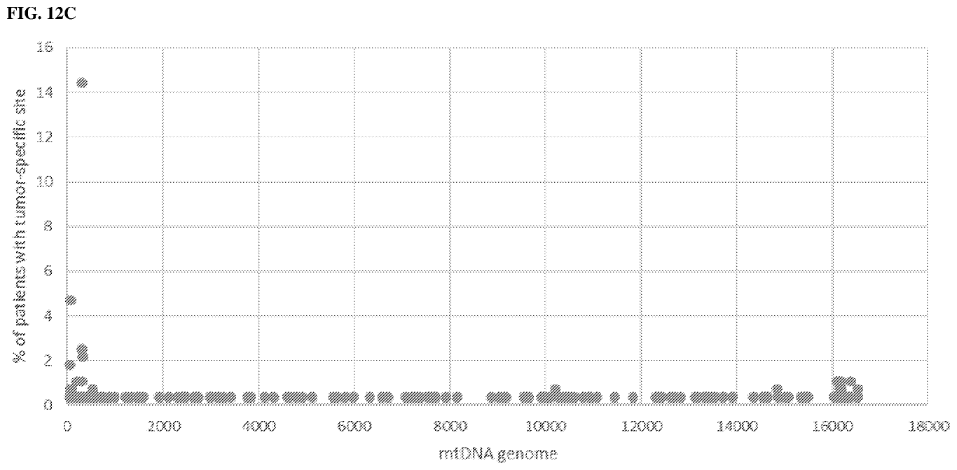

The consensus sequences from tumor and blood differ at 169 sites (“tumor-specific” sites) scattered across the entire genome (FIG. 12C). Mutations at these sites (“tumor-specific” mutations) were, however, present at low frequency in the blood of 7.03% of patients and in 18.95% of patients in normal liver. Most of the tumor-specific mutations (88.16%) were found only once in other LC patients. Only one tumor-specific mutation at site 310 is present in 14.44% of the LC patients (FIG. 12A). Both observations indicate a low association of these mutations with LC.

mtDNA in LC and NC Patients' Blood

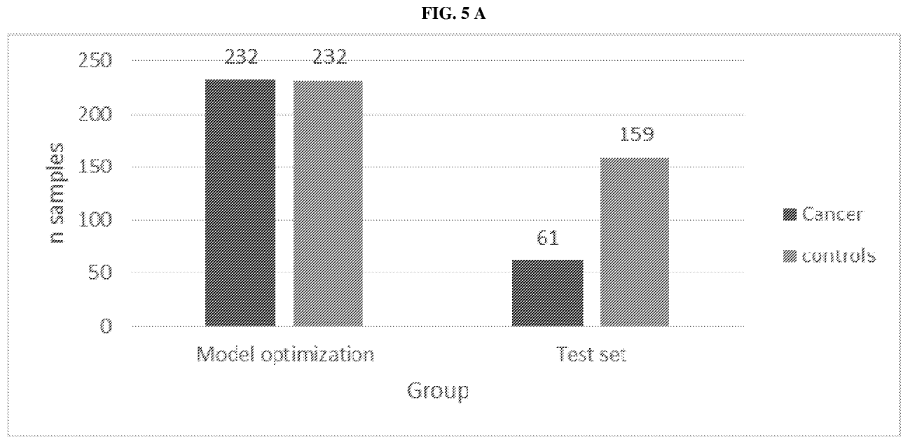

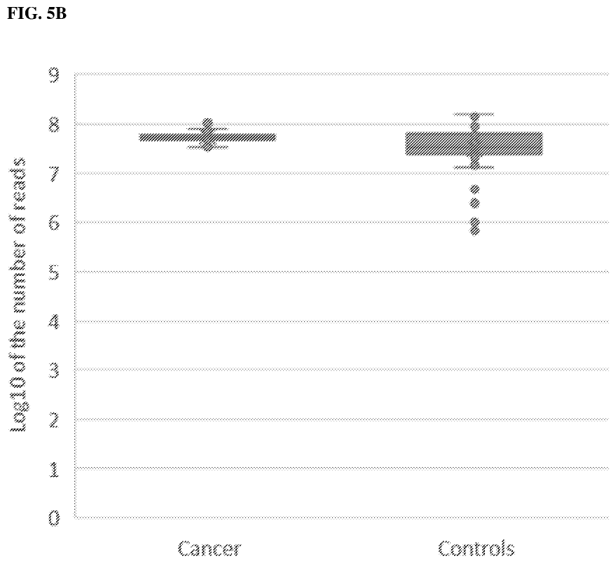

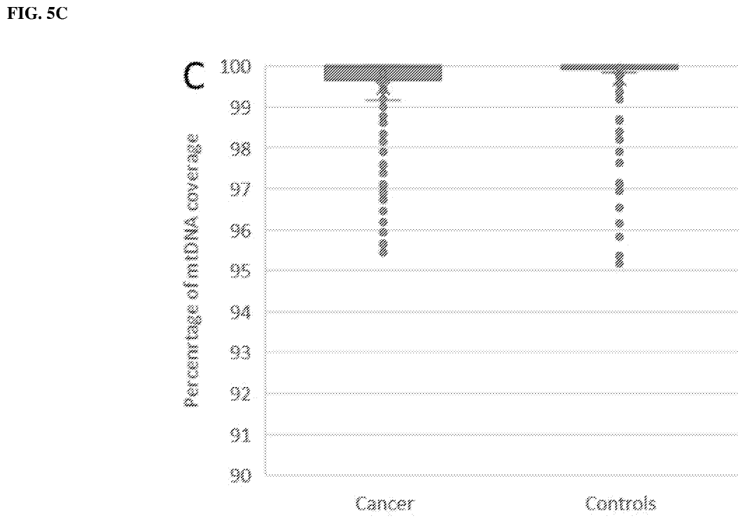

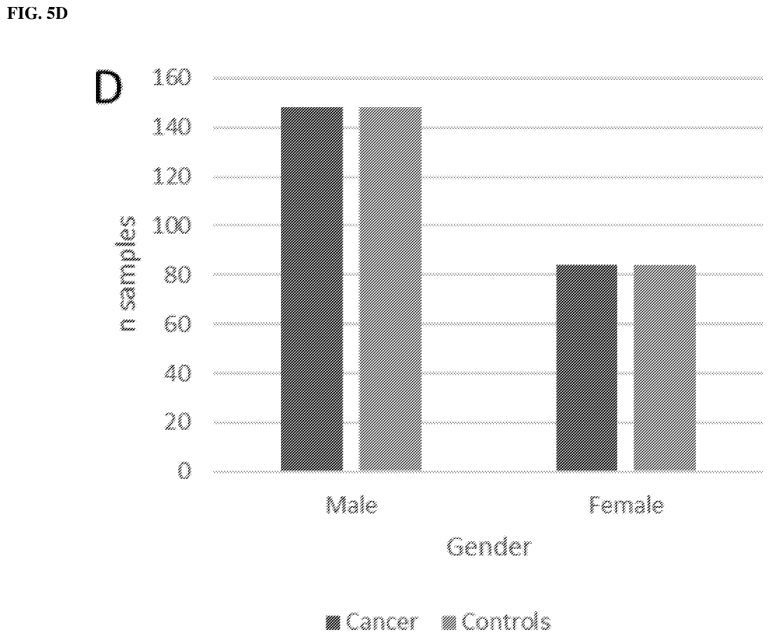

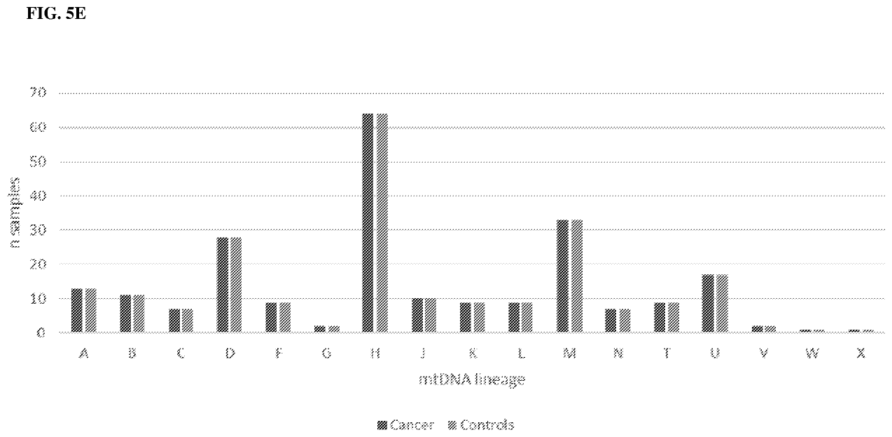

Considering that mtDNA was tested in blood from all cases studied here, analyses on genetic differences in mtDNA between LC and NC were focused on data from blood. FIG. 5 shows distribution of the number of available samples, gender and mtDNA lineages between the LC and NC groups. All these factors were equalized to ensure statistical significance of observations on differences between these two groups. The two groups showed small but statistically significant differences in average entropy of mtDNA, percentage of exome reads mapped to mtDNA, percentage of all reads mapped to mtDNA, and number of polymorphic sites (Table 2).

TABLE 2
Comparison between LC and NC samples
Ratio of the averages and p value of the paired samples t-test.

| | LC | NC | Ratio | p value |
|---|---|---|---|---|
| Number of reads (log10) | 7.7429 | 7.4892 | 0.9672 | 2.62E−15 |
| mtDNA average depth | 49.6176 | 120.0060 | 2.4186 | 1.73E−12 |
| mtDNA total entropy | 0.0011 | 0.0014 | 1.2798 | 1.96E−05 |
| Percentage of the mtDNA | 99.4562 | 99.6885 | 1.0023 | 0.004217 |
| genome covered | | | | |
| Percentage of all reads that | 0.0073 | 0.0388 | 5.3349 | 2.78E−30 |
| map to the mtDNA genome | | | | |
| Number of polymorphic | 68.4334 | 129.8362 | 1.8973 | 5.86E−06 |
| sites. | | | | |

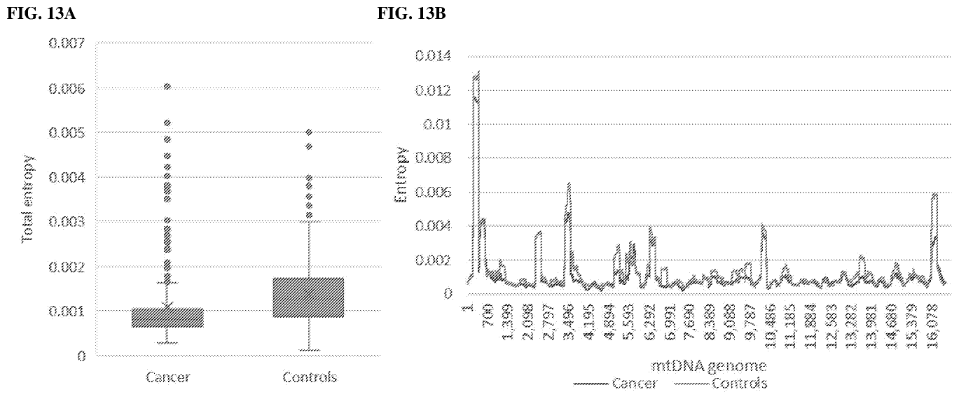

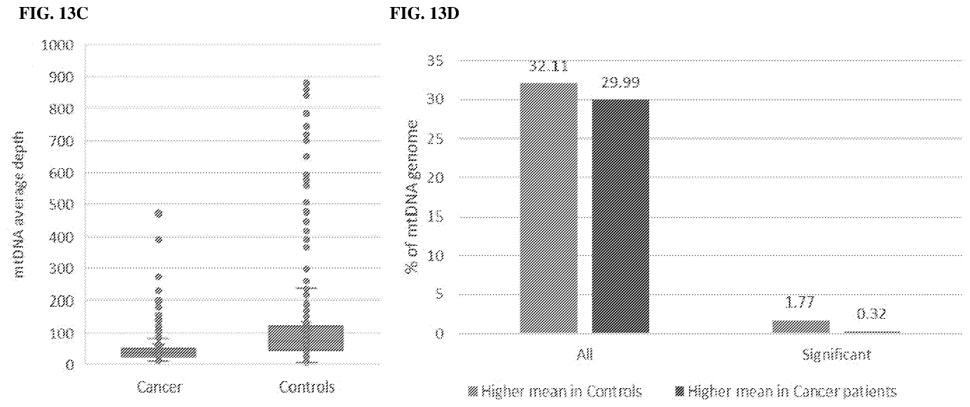

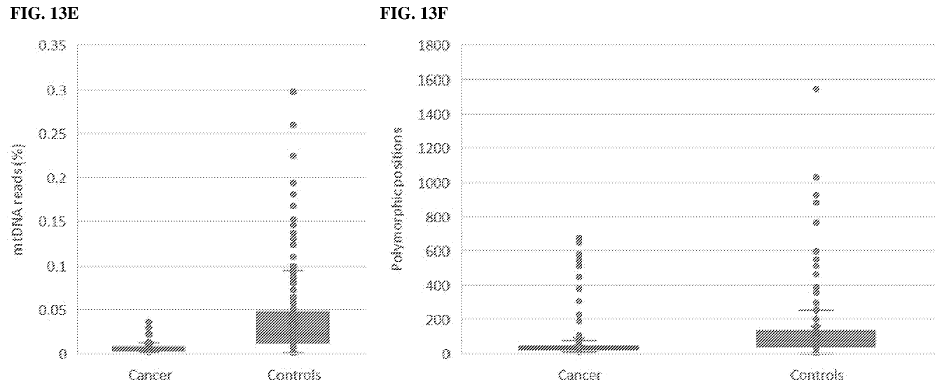

When compared with NC, LC have 1.24-times lower average total entropy (p=2.84E-47) and 3.6-times lower percentage of all reads mapped to mtDNA (p=8.23E-19) (Table 2 and FIG. 13). Among all mtDNA polymorphic sites, 2.09% showed a significantly different mean entropy between LC and NC. These selected sites were distributed across the entire mtDNA evenly. Only 0.32% of the sites had a higher mean entropy (p<0.05) but 1.77% had a lower mean entropy in LC (p<0.05). Thus, certain polymorphic sites scattered along mtDNA differ in the degree of diversity between LC and NC patients, indicating their potential application as markers of LC.

Genetic Association with LC

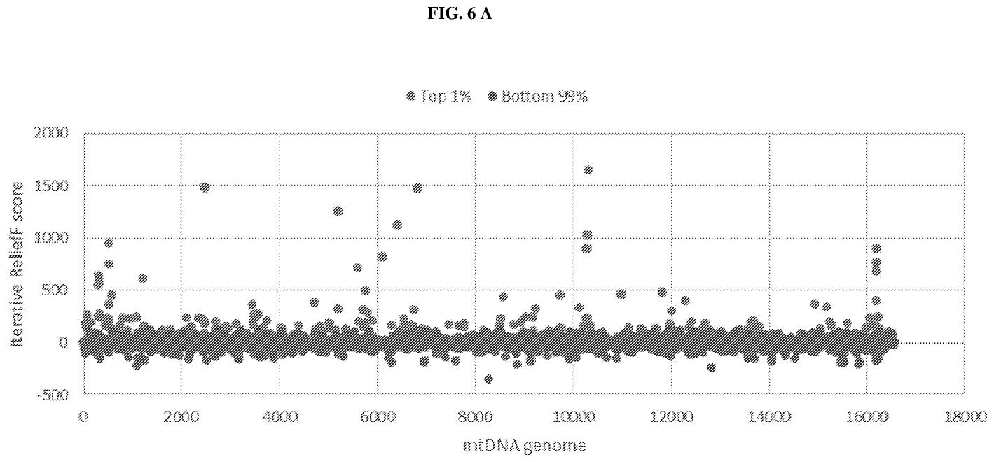

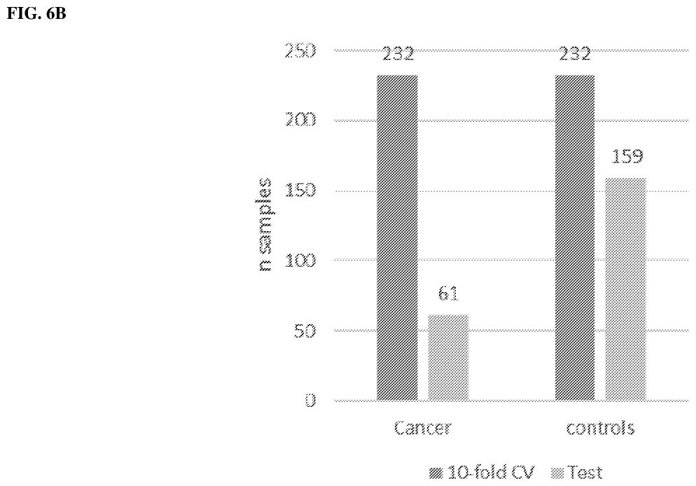

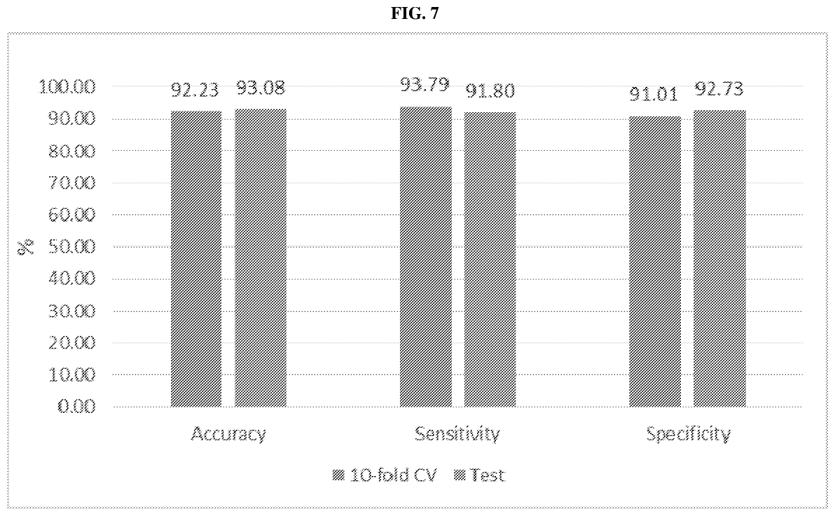

The top 1% of the mtDNA 16,569 nucleotide sites (n=166) with the highest Iterative Relief scores were used for the classifier optimization (FIG. 6A). These sites are not clustered in any gene but spread over mtDNA. The samples were separated into two groups, the first was used for the classifier optimization in 10-fold Cross-Validation (10×CV), and the second, which was not used for the optimization, was used for the final classifier testing. FIG. 6B shows the number of samples in each set. Using the first set, the RF-based classifier showed accuracy of up to 99.78% and an average accuracy in 10×CV of 92.23%. Finally, the RF classifier yielded an accuracy of 93.08% on the test dataset (FIGS. 6 and 7). All these data indicate that the mtDNA heterogeneity is strongly associated with LC and NC.

Among the top 1% LC-specific sites (n=166) selected by ReliefF, only 11 (6.6%) are shared with the “tumor-specific” sites (n=169) selected using consensus sequences. Thus, although both are scattered across the entire mtDNA, individual sites from both groups are very different. A number of machine learning algorithms were tested. Random forest [14] provided the best performance.

HVS1 Association with LC

Taking into consideration that polymorphic sites of significance found here are distributed along the entire mtDNA, we tested one of the most heterogeneous mitochondrial genomic regions, HVS1 at position 15,977-16,391 bp, which has been extensively used in many genetic studies. Reads covering this region were extracted from the exome data and used to generate the RF classifier. The average 10×CV accuracy was 83.22%, indicating that, although at the reduced rate, the distribution of sites' entropy in this region alone is strongly associated with LC. However, it should be noted that increase in the coverage depth might help to identify more polymorphic sites in this region, thus potentially improving accuracy of classification. Application of UDS to a small genomic region offers a greater control over sequencing depth, which is important for accurate assessment of genetic heterogeneity, especially in mtDNA extracted from blood where its concentration is low.

A high-throughput multiplexing MiSeq HVS1 protocol was used to test 136 blood samples obtained from HCV infected persons who were not tested for cancer. The amplicon UDS data were pre-processed using the same pipeline that was applied to the exome data. In average, 383,984 reads were sequenced per patient, with each read covering the entire HVS1. Among 433 sites sequenced from HVS1, 96.57% of sites in average were polymorphic, which is significantly greater of 0.63% and 1.32% of polymorphic sites found in the exome data from LC and NC, correspondingly.

Assessment of the Neoplasm Histologic Grade

In the dataset presented in this Example, 41 samples were classified as stage 1 cancer, 134 samples were stage 2, 104 samples were stage 3 and 13 samples were stage 4 (FIG. 10C). In 10×CV, the RF Regression yielded an average absolute error of 0.61, which is only 2× better than the average absolute error of a random assignment (1.249), showing only a moderate association of SMEPs with the grades. Implementation of binary classification schemes instead of regression (e.g. Stage 1 vs all others) didn't improve classification accuracy.

### DISCUSSION

Analyses conducted here indicate that heterogeneity profiles of the intra-host mtDNA variants from blood are strongly associated with LC. Although cancer detection is usually focused on genetic analysis of nuclear DNA, mtDNA has been shown to be functionally associated with several cancer types. Owing to its clonal nature, high copy number and high mutation rate, mtDNA has many practical advantages over nuclear DNA in application to the cancer detection. Mitochondria supply energy for all metabolic processes and control apoptosis, and as such are essential for multiplication of cancer cells. The mitochondrial oxidative phosphorylation system has a major effect on tumor progression. In addition, enhanced progression to malignancy was observed in cells with compromised mitochondrial integrity. mtDNA mutations are significantly associated with the development of various types of cancer.

Clonal expansion of mutant mtDNA species was reported in 27%-80% (in average 54%) of malignant tumor samples. In concert with this observation, we found that consensus sequences of mtDNA differ between tumor and blood from ˜58% of patients. Both particle-associated and free mtDNA are present in blood, providing a convenient and minimally invasive way for the detection of cancer-related mitochondrial mutants. As many cancer types, LC is associated with clonally expanding mtDNA mutations. The clonal expansion should affect genetic composition of mtDNA variants in blood. However, such an effect is not straightforward because mtDNA in blood has a very complex origin. Moreover, requirements for efficient energy supply to rapidly replicating malignant cells constrains genetic composition of mitochondria in tumors.

The clonal expansion and genetic constraints coupled with a small size (16,569 bp) make mtDNA especially suitable for the accurate assessment of association of intra-host genetic heterogeneity, rather than specific mutations, with cancer. Application of heterogeneity profiles implemented here to the LC detection overcomes the often-idiosyncratic presentation of specific mutations in cancer. Indeed, most tumor-specific variants (99.4%) found in this study were present in less than 5% of LC patient, thus impeding their use as general cancer markers. Complex and variable genetic nature of cancer is well established. It hinders the identification of specific mutations suitable for cancer diagnostics. However, measures of intra-host genetic diversity in place of specific states of nucleotide sites mitigate the contribution of host-specific genetics to the detection of associations with cancer.

Tumor-specific mutations were present at low frequency in the blood of only 7.03% of patients. This finding indicates that the direct contribution of tumor to the genetic composition of mtDNA in blood is limited, thus potentially confounding the detection of tumor-specific genetic variants in blood for cancer diagnostics. This concern becomes especially relevant when one considers a significant drop in mtDNA load in blood observed in this study and also reported elsewhere. Nevertheless, the RF-classifiers generated here separated LC and NC patients with accuracy exceeding 93%, indicating the existence of a strong LC-specific genetic signal in intra-host mtDNA populations.

Genetic factors used in the RF-classifiers are fundamentally different from tumor-specific mutations identified from consensus mtDNA sequences. Only 11 tumor-specific sites were among the top 166 sites selected by entropy as relevant to the LC/NC classification, despite the fact that both sets of sites scattered along the entire mtDNA. Site entropy or its Z-score do not have information on a specific nucleotide state of a site, rather both measure nucleotide diversity at each site, thus reducing strong effects of specific mutations on associations captured by our models. There are many genetically diverse lineages of mtDNA. Although the LC and NC datasets were matched by geographic location and mtDNA lineages, genetic differences among different genetic types of mtDNA may impede the identification of cancer-specific mutations, especially in a limited dataset. Entropy, however, represents a more general genetic information that can adequately trim genetic differences among mtDNA lineages, focusing nucleotide heterogeneity analyses on the identification of other than lineage-specific traits. Models generated using Z-scores performed as well as the entropy-based models. However, contribution of standardization achieved by application of Z-score to accuracy of models may become more apparent on more heterogeneous datasets.

Detection of associations can be obtained using different analytical techniques. In this Example, machine-learning algorithms were applied to extract genetic information from mtDNA for discriminating between LC and NC. Machine learning has many important advantages over other techniques and allows for the rapid and reliable identification of complex patterns in data. Application of the algorithms is routine in industrial and technological applications and only recently became successfully explored in clinical field. Machine learning presents a new opportunity to cancer diagnostics by shortcutting research from learning molecular mechanisms before developing applications to direct identification of reliable markers, thus accelerating development of accurate cancer detection.

This Example shows that tumor-specific mutant mtDNA species may be present at a very low concentration in blood. The detection of such minority variants can be achieved by UDS. Indeed, UDS has been applied to the efficient detection of tumor DNA and to the detection of minority cancer-specific DNA variants. However, a significant depletion of mtDNA reported for several cancer types such as bladder, breast, kidney, and liver cancer makes the detection of minority tumor-specific variants especially challenging. The observed ˜2-fold decline in the number of reads mapped to mtDNA from tumor as compared to normal liver tissue adds to this report and further emphasizes potential difficulties in identification of specific mutant variants in tested blood. These observations indicate that consistent detection of minority variants is strongly contingent to a very high depth of sequencing. However, in difference to the detection of specific mutations, accurate estimation of site heterogeneity can be done at a moderate sequencing depth, thus providing a more reliable source of cancer-specific markers.

Uniform and adequate read coverage of the entire mtDNA can be challenging for the shotgun-based UDS. Sequencing of a single amplicon offers a greater control over the read coverage. However, it limits the mtDNA presentation to a single genomic region. Distribution of the nucleotide sites most contributing to the LC association across the entire mtDNA indicates, though, that many individual genomic regions contribute to such association. Coevolution among genomic sites is one of the most important properties of genetic systems. Epistatic connectivity modelled using individual viral genomic regions was found to be strongly associated with such traits as disease progression, drug resistance, and host specificity. It has been shown that, owing to the uneven distribution of the modelled epistatic connections, certain genomic regions contribute more than the other to such associations. Taking these observations in consideration, it is hypothesized that such highly heterogeneous region of mtDNA as HVS1, may have sufficient genetic information to identify association with LC. Indeed, the model constructed using HVS1 alone identified LC vs NC with 83.22% 10×CV accuracy, indicating its applicability to the detection of LC. Genetic analysis of the single amplicon UDS data revealed a much greater HVS1 heterogeneity, with 96.57% of HVS1 sites being polymorphic. The increase in diversity identified by the amplicon-based UDS warrants further investigation on the detection of the HVS1 association with LC. The data presented in this Example indicate significant differentiation of mtDNA heterogeneity between LC and NC patients.

The method described herein has three important advantages:

1. It limits the search to the small mtDNA genome (16569 bases), which has already been functionally associated with several cancer types. Due to mtDNA's clonal nature, high copy number and high mutation rate, its use provides several practical laboratory advantages, lowering cost and increasing sensitivity. Mitochondria are very important cell organelles as they produce energy supply for cellular processes via the oxidative phosphorylation system. Functional mitochondria are essential for the cancer cell and it has been shown both in vitro and in vivo that the oxidative phosphorylation system can have a major influence on tumor progression and that when mitochondrial integrity is compromised cancer progression is enhanced [16] [17].

Several studies have suggested an eminent role for mitochondrial DNA (mtDNA) mutations in the development of a wide variety of cancer types (for a review see [17]). In more than 20 studies, the percentage of samples with clonally expanded mtDNA mutations ranged from 27% to almost 80%, averaging 54% (for a review see [18]). More recently, Ju et al [19] found that, in contrast to the mutational signatures found in nuclear genomes, where there is striking heterogeneity both across tumor types and across individuals within a tumor type (Alexandrov et al., 2013), the mutational profile in the mitochondrial genome of somatic cells is remarkably homogeneous. More recently, a number of mtDNA mutations have shown considerable heterogeneity across tumor types [19].

By virtue of their clonal nature, high copy number and high mutation rate [20], mtDNA mutations provide a powerful marker for noninvasive detection of cancer. Both particle-associated and free mtDNA are known to be present in plasma [21], a phenomenon that is being exploited since the early efforts to detect cancer-related mitochondrial mutants in serum [3], even before the advent of UDS. Given that liver cancer has been associated with clonally expanded mtDNA mutations [22-28], this cancer type was chosen as the classifier's proof of concept. However, given that mtDNA mutations have an eminent role in the development of a wide variety of cancer types, future work includes the application of the method to other cancer types were mtDNA mutations have been observed.

2. It doesn't rely on a particular target nucleotide but rather on its genetic heterogeneity. If the actual nucleotides were used, the classification accuracy would likely be higher, but its generalization power to the general population would likely be lower. This is due to the fact that there are many genetic differences between mtDNA genomes that have accumulated during the history of human population, differences that could erroneously separate our particular set of cancer and control samples. Although some precautions were taken to avoid this by matching the geographic region and mtDNA lineage distribution of the cancer and control samples, this is just a subset of the total human mtDNA variation. For this reason, the genetic heterogeneity of each position was used, rather than its particular target nucleotide, thus increasing the generalization ability of the test by essentially ignores the differences between mtDNA lineages that exist between individuals and human populations. Another step in this direction is the calculation of Z-scores, which makes the profiles more comparable, and thus increases the generalization power of the test, as shown by its better performance on cross-validation (data not shown).

3. The salient features of the heterogeneity profile are found with machine learning, increasing the generalization ability of the test. Machine learning is routinely used in industry and technological applications, but its huge potential for the clinical field is just recently being explored [29, 30]. During the last decade, research on the actual cellular and genomic mechanisms of cancer development has taken priority over the search of diagnostic biomarkers, the rationale being that a fully understood mechanism can then be used to detect cancer. However, the methods described herein do not require a full understanding of the complex association of mtDNA and cancer for using its huge diagnostic potential. In addition, machine learning provides a fully automated report that does not require a subject matter expert, providing at the end a probability that the sample comes from a cancer patient that can be used by medical practitioners like any other laboratory test.

In conclusion, the diagnostic method described herein accurately distinguishes cancer from healthy samples using mtDNA heterogeneity profiles obtained from exome sequencing of blood samples. The rapid, cheap and non-invasive nature of the classifier described herein could bring a fundamental change to cancer screening programs.

### REFERENCES

1. Organization, W.H. Cancer. 2016 February 2015 January 2016]; Available from: http://www.who.int/mediacentre/factsheets/fs297/en/
2. Larrea, E., et al., New Concepts in Cancer Biomarkers: Circulating miRNAs in Liquid Biopsies. Int J Mol Sci, 2016. 17(5).
3. Fliss, M. S., et al., Facile detection of mitochondrial DNA mutations in tumors and bodily fluids. Science, 2000. 287(5460): pp. 2017-9.
4. Yong, E., Cancer biomarkers: Written in blood. Nature, 2014. 511(7511): pp. 524-6.
5. Andrews, R. M., et al., Reanalysis and revision of the Cambridge reference sequence for human mitochondrial DNA. Nat Genet, 1999. 23(2): p. 147.
6. Calabrese, C., et al., MToolBox: a highly automated pipeline for heteroplasmy annotation and prioritization analysis of human mitochondrial variants in high-throughput sequencing. Bioinformatics, 2014. 30(21): pp. 3115-7.
7. Picardi, E. and G. Pesole, Mitochondrial genomes gleaned from human whole-exome sequencing. Nat Methods, 2012. 9(6): pp. 523-4.
8. Lo, C. C. and P. S. Chain, Rapid evaluation and quality control of next generation sequencing data with FaQCs. BMC Bioinformatics, 2014. 15: p. 366.
9. Morelli, M. J., et al., Evolution of foot-and-mouth disease virus intra-sample sequence diversity during serial transmission in bovine hosts. Vet Res, 2013. 44: p. 12.
10. Shannon, C., A Mathematical Theory of Communication Bell Syst Tech J, 1948. 27(379-423).
11. The Cancer Genome Atlas. [cited February 2016; Available from: http://cancergenome.nih.gov/.
12. Genomes Project, C., et al., A global reference for human genetic variation. Nature, 2015. 526(7571): pp. 68-74.
13. Robnik-Sikonja, M. and I. Kononenko, Theoretical and Empirical Analysis of ReliefF and RReliefF. Machine Learning Journal, 2003. 53: pp. 23-69.
14. Breiman, L., Random Forests. Machine Learning, 2001. 45(1): pp. 5-32.
15. Pedregosa, F., et al., Scikit-learn: Machine learning in Python. Journal of Machine Learning Research, 2011. 12(October): pp. 2825-2830.
16. Wallace, D. C., Mitochondria and cancer. Nat Rev Cancer, 2012. 12(10): pp. 685-98.
17. van Gisbergen, M. W., et al., How do changes in the mtDNA and mitochondrial dysfunction influence cancer and cancer therapy? Challenges, opportunities and models. Mutat Res Rev Mutat Res, 2015. 764: pp. 16-30.
18. Khaidakov, M. and R. J. Shmookler Reis, Possibility of selection against mtDNA mutations in tumors. Mol Cancer, 2005. 4: p. 36.
19. Ju, Y. S., et al., Origins and functional consequences of somatic mitochondrial DNA mutations in human cancer. Elife, 2014. 3.
20. Wallace, D. C., Mitochondrial DNA Variation in Human Radiation and Disease. Cell, 2015. 163(1): pp. 33-8.
21. Chiu, R. W., et al., Quantitative analysis of circulating mitochondrial DNA in plasma. Clin Chem, 2003. 49(5): pp. 719-26.
22. Wong, L. J., et al., Molecular alterations in mitochondrial DNA of hepatocellular carcinomas: is there a correlation with clinicopathological profile? J Med Genet, 2004. 41(5): p. e65.
23. Nishikawa, M., et al., Somatic mutation of mitochondrial DNA in cancerous and noncancerous liver tissue in individuals with hepatocellular carcinoma. Cancer Res, 2001. 61(5): pp. 1843-5.
24. Tamori, A., et al., Correlation between clinical characteristics and mitochondrial D-loop DNA mutations in hepatocellular carcinoma. J Gastroenterol, 2004. 39(11): pp. 1063-8.
25. Wheelhouse, N. M., et al., Mitochondrial D-loop mutations and deletion profiles of cancerous and noncancerous liver tissue in hepatitis B virus-infected liver. Br J Cancer, 2005. 92(7): pp. 1268-72.
26. Nomoto, S., et al., Mitochondrial D-loop mutations as clonal markers in multicentric hepatocellular carcinoma and plasma. Clin Cancer Res, 2002. 8(2): pp. 481-7.
27. Zhang, R., et al., Identification of sequence polymorphism in the D-Loop region of mitochondrial DNA as a risk factor for hepatocellular carcinoma with distinct etiology. J Exp Clin Cancer Res, 2010. 29: p. 130.
28. Shawky, R., et al., Mitochondrial alterations in children with chronic liver disease. Egyptian Journal of Medical Human Genetics, 2010. 1(2): pp. 143-151.
29. Kononenko, I., Machine learning for medical diagnosis: history, state of the art and perspective. Artif Intell Med, 2001. 23(1): pp. 89-109.
30. Foster, K. R., R. Koprowski, and J. D. Skufca, Machine learning, medical diagnosis, and biomedical engineering research—commentary. Biomed Eng Online, 2014. 13: p. 94.

1. A method of diagnosing a cancer in a patient, comprising:
(a) providing a heterogeneity profile of mitochondrial DNA (mtDNA) obtained from a sample from a patient, wherein the heterogeneity profile comprises a level of genetic heterogeneity quantified at one or more nucleotide positions of a mtDNA genome;
(b) classifying the patient heterogeneity profile as cancer-positive or cancer-negative based on the result of a machine learning classifier; and
(c) diagnosing the presence or absence of cancer in the patient based on the classification.

2. The method of claim 1, wherein the machine learning algorithm is a random forest, which has been trained with a data set comprising heterogeneity profiles of mtDNA genomes from positive control subjects diagnosed with the cancer and heterogeneity profiles of mtDNA genomes from negative control subjects diagnosed as not having the cancer;

3. The method of claim 2, wherein the level of genetic heterogeneity at each nucleotide position of the mtDNA genome from each patient and control subject is quantified by calculating Shannon entropy.

4. The method of claim 3, wherein the level of genetic heterogeneity at each nucleotide position is quantified by transforming the Shannon entropy level into a Z-score.

5. The method of claim 1, wherein the cancer is liver cancer.

6. The method of claim 5, wherein the liver cancer is hepatocellular carcinoma.

7. The method of claim 2, wherein the heterogeneity profiles comprise levels of entropy quantified at each nucleotide position of the mtDNA genomes of the patient, the positive control subjects, and the negative control subjects, and wherein the mtDNA genomes are sequenced using next-generation sequencing.

8. A system comprising:
a memory; and
at least one computing device to:
obtain heterogeneity profile data including data quantifying a level of genetic heterogeneity at one or more nucleotide positions of a mitochondrial DNA (mtDNA) genome from a sample corresponding to a patient;
execute a machine learning classifier to analyze the patient heterogeneity profile, wherein the machine learning classifier has been trained with data including first heterogeneity profile data indicative of presence of a cancer and second heterogeneity profile data indicative of absence of the cancer; and,
based on the prediction of the machine learning classifier, store, in the memory, an indication of presence or absence of the cancer corresponding to the patient.

9. The system of claim 8, wherein the machine learning classifier is a random forest.

10. The system of claim 8, wherein the first heterogeneity profile data comprise levels of genetic heterogeneity quantified at one or more nucleotide positions in mtDNA genomes from positive control subjects diagnosed with the cancer, and the second heterogeneity profile data comprise levels of genetic heterogeneity quantified at one or more nucleotide positions of mtDNA genomes from negative control subjects diagnosed as not having the cancer.

11. The system of claim 10, wherein the level of genetic heterogeneity at each nucleotide position of the mtDNA genome from each patient and control subject is quantified by calculating Shannon entropy.

12. The system of claim 11, wherein the level of genetic heterogeneity at each nucleotide position is quantified by transforming the Shannon entropy level into a Z-score.

13. The system of claim 8, wherein the cancer is liver cancer.

14. The system of claim 13, wherein the liver cancer is hepatocellular carcinoma.


SEQUENCE LISTING (2 sequences)

SEQ ID NO: 1 - DNA, 20 bp, Homo sapiens
caccattagcacccaaagct

SEQ ID NO: 2 - DNA, 20 bp, Homo sapiens
gaggatggtggtcaagggac